(19) United States
(12) Patent Application Publication
(10) Pub. No.: US 2022/0238186 A1
(43) Pub. Date: Jul. 28, 2022

(54) Sequence Alignment Method and System

(71) Applicant: Inspur Suzhou Intelligent Technology Co., Ltd., Suzhou (CN)

(72) Inventors: Jian Zhao, Suzhou (CN); Hongzhi Shi, Suzhou (CN); Xingchen Cui, Suzhou (CN)

(21) Appl. No.: 17/615,580

(22) PCT Filed: Oct. 31, 2019
(86) PCT No.: PCT/CN2019/114531

§ 371 Date: Nov. 30, 2021

(30) Foreign Application Priority Data
Aug. 23, 2019 (CN) 201910796168.0

(51) Int. Cl. G16B 30/10; G16B 50/30

(52) U.S. Cl. CPC G16B 30/10; G16B 50/30

(57) ABSTRACT

Provided are a sequence alignment method and system. The method comprises: searching for candidate alignment locations of all seeds in a sequence to be aligned, and after performing normalization processing on the candidate alignment locations of all seeds, acquiring the longest seeds of various types in a bitmap mode; and then filtering out all seeds covered by the longest seeds, so that the number of candidate comparison locations needing to be aligned subsequently is reduced. Therefore, the workload of subsequent alignment work is greatly reduced, the sequence alignment speed is increased, and meanwhile, the alignment precision is guaranteed.

The present application claims the priority of Chinese patent application filed with the China Patent Office on Aug. 23, 2019, with the application number of 201910796168.0 and entitled “Sequence Alignment Method and System”, the contents of which are incorporated herein by reference in its entirety.

### TECHNICAL FIELD

The present invention relates to the technical field of computers, in particular to a sequence alignment method and system.

### BACKGROUND

With the increasing maturity of the biological gene detection technology, gene sequence alignment can be achieved through extracting personal genes, to predict the possibility of suffering from multiple diseases, and lock the genes of personal diseases for prevention and treatment in advance.

The existing sequence alignment methods include two stages: seed lookup and seed alignment, a series of subsequences on the read sequence to be aligned are extracted, that is, the seed, and then the candidate alignment location (CAL) table is searched, to find out the exact matching location of each seed on the reference sequence, and then the base at the matching location is read and compared with read. In order to improve the accuracy of sequence alignment, it is necessary to find out the location of the seed of the sequence to be aligned read in the reference sequence as much as possible, therefore, the length of the seed is usually shorter. However, such a seed will hit a lot of times on the reference sequence, and the performance of sequence alignment of the existing processor is limited, the sequence alignment speed is slow, and the requirement of fast or real-time acquisition of gene alignment results cannot be satisfied.

### SUMMARY OF THE INVENTION

The present invention provides a sequence alignment method and system, to solve the problems in the prior art that a seed will hit a lot of times on the reference sequence, and the performance of sequence alignment of the existing processor is limited, the sequence alignment speed is slow, and the requirement of fast or real-time acquisition of gene alignment results cannot be satisfied.

To achieve the above objective, the present invention provides the following technical solution:

a sequence alignment method includes:

searching for all the seeds in a sequence to be aligned, searching for a candidate alignment location table according to the seeds, and determining candidate alignment locations of all the seeds on a reference sequence;

normalizing the candidate alignment locations of all the seeds on the reference sequence according to the locations of all the seeds in the sequence to be aligned, to obtain normalized candidate alignment locations of all the seeds;

selecting the longest seeds of all the species by means of a bitmap according to the normalized candidate alignment locations;

filtering out all the seeds covered by the longest seeds of all the species to obtain filtered seeds; and

aligning the filtered seeds with the candidate alignment locations corresponding to each seed in the filtered seeds to obtain a sequence alignment result.

Optionally, the selecting the longest seeds of all the species by means of a bitmap according to the normalized candidate alignment locations includes:

setting the candidate alignment locations of all the seeds on the reference sequence to 1 by means of a bitmap according to the normalized candidate alignment locations, setting locations on the reference sequence other than the candidate alignment locations to 0, selecting the longest consecutive 1 corresponding seeds at different starting locations in the bitmap, to obtain the longest seeds of all the species.

Optionally, after filtering out all the seeds covered by the longest seeds of all the species to obtain filtered seeds, the sequence alignment method further includes:

counting the number of occurrences of each longest seed in the reference sequence;

judging whether the number of occurrences of each longest seed in the reference sequence is less than a first preset threshold;

if it is judged that the number of occurrences of any of the longest seeds in the reference sequence is less than a first preset threshold, then splitting from the longest seed a seed containing a base in the middle location of the longest seed; and

if it is judged that the number of occurrences of each of the longest seeds in the reference sequence is greater than or equal to the first preset threshold, then performing the step of aligning the filtered seeds with the candidate alignment locations corresponding to each seed in the filtered seeds to obtain the sequence alignment results.

Optionally, the normalizing the candidate alignment locations of all the seeds on the reference sequence according to the locations of all the seeds in the sequence to be aligned to obtain normalized candidate alignment locations of all the seeds includes:

according to the locations of all the seeds in the sequence to be aligned, normalizing the candidate alignment locations of all the seeds on the reference sequence to a candidate alignment location on the reference sequence corresponding to the starting location of the sequence to be aligned to obtain the normalized candidate alignment locations of all the seeds.

Optionally, after determining the candidate alignment locations of all the seeds on the reference sequence, the sequence alignment method further includes:

judging whether the number of candidate alignment locations of each seed on the reference sequence exceeds a second preset threshold;

if it is judged that the number of candidate alignment locations of any seed on the reference sequence exceeds the second preset threshold, then selecting the candidate alignment locations for subsequent alignment from all the candidate alignment locations of the seed according to a preset number of intervals; and

if it is judged that the number of candidate alignment locations of each seed on the reference sequence exceeds a second preset threshold, then performing the step of normalizing the candidate alignment locations of all the seeds on the reference sequence according to the locations of all the seeds in the sequence to be aligned to obtain the normalized candidate alignment locations of all the seeds.

A sequence alignment system includes:

a determination unit, configured to search for all the seeds in a sequence to be aligned, search for a candidate alignment location table according to the seeds, and determine candidate alignment locations of all the seeds on a reference sequence;

a processing unit, configured to normalize the candidate alignment locations of all the seeds on the reference sequence according to the locations of all the seeds in the sequence to be aligned, to obtain normalized candidate alignment locations of all the seeds;

a selection unit, configured to select the longest seeds of all the species by means of a bitmap according to the normalized candidate alignment locations;

a filtering unit, configured to filter out all the seeds covered by the longest seeds of all the species to obtain filtered seeds; and

an alignment unit, configured to align the filtered seeds with the candidate alignment locations corresponding to each seed in the filtered seeds to obtain a sequence alignment result.

Optionally, the selection unit is configured to set the candidate alignment locations of all the seeds on the reference sequence to 1 by means of a bitmap according to the normalized candidate alignment locations, set locations on the reference sequence other than the candidate alignment locations to 0, select the longest consecutive 1 corresponding seeds at different starting locations in the bitmap, to obtain the longest seeds of all the species.

Optionally, the sequence alignment system further includes:

a statistical unit, configured to count the number of occurrences of each longest seed in the reference sequence;

a first judgment unit, configured to judge whether the number of occurrences of each longest seed in the reference sequence is less than a first preset threshold;

if it is judged that the number of occurrences of any of the longest seeds in the reference sequence is less than a first preset threshold, then splitting from the longest seed a seed containing a base in the middle location of the longest seed; and

if it is judged that the number of occurrences of each of the longest seeds in the reference sequence is greater than or equal to the first preset threshold, then performing the step of aligning the filtered seeds with the candidate alignment locations corresponding to each seed in the filtered seeds to obtain the sequence alignment results.

Optionally, the processing unit is configured to, according to the locations of all the seeds in the sequence to be aligned, normalize the candidate alignment locations of all the seeds on the reference sequence to a candidate alignment location on the reference sequence corresponding to the starting location of the sequence to be aligned to obtain the normalized candidate alignment locations of all the seeds.

Optionally, the sequence alignment system further includes:

a second judgment unit, configured to judge whether the number of candidate alignment locations of each seed on the reference sequence exceeds a second preset threshold;

if it is judged that the number of candidate alignment locations of any seed on the reference sequence exceeds the second preset threshold, then selecting the candidate alignment locations for subsequent alignment from all the candidate alignment locations of the seed according to a preset number of intervals; and

if it is judged that the number of candidate alignment locations of each seed on the reference sequence exceeds a second preset threshold, then performing the step of normalizing the candidate alignment locations of all the seeds on the reference sequence according to the locations of all the seeds in the sequence to be aligned, to obtain the normalized candidate alignment locations of all the seeds.

It can be known from the above technical solution that, the present invention discloses a sequence alignment method and system, wherein the candidate alignment locations of all the seeds in the sequence to be aligned are searched for, after normalizing the candidate alignment locations of all the seeds, the longest seeds of various species are acquired through the manner of bitmap, and then all the seeds covered by the longest seeds are filtered out, to reduce the number of candidate alignment locations needing to be aligned subsequently, so as to greatly reduce the workload of subsequent alignment work, and improve the sequence alignment speed and ensure alignment accuracy.

### BRIEF DESCRIPTION OF THE DRAWINGS

In order to more clearly illustrate the technical solutions in the embodiments of the present application or the prior art, the accompanying drawings to be used in the description of the embodiments or the prior art will be briefly introduced, apparently, the accompanying drawings in the following description are merely embodiments of the present invention, and other drawings can be obtained from the provided drawings without any creative effort by those skilled in the art.

FIG. 1 is a flow chart of a sequence alignment method disclosed in the embodiment of the present invention;

FIG. 2 is a schematic diagram of a candidate alignment location of seeds in the sequence to be aligned in the embodiment of the present invention on the reference sequence;

FIG. 3 is a schematic diagram of filtered seeds in the embodiment of the present invention;

FIG. 4 is a schematic diagram of seeds containing a base in the middle location of the longest seed and split from the final seeds; and

FIG. 5 is a schematic diagram of a sequence alignment system disclosed in the embodiment of the present invention.

### DETAILED DESCRIPTION OF THE EMBODIMENTS

The technical solutions in the embodiments of the present invention will be described clearly and completely below in combination with the accompanying drawings in the embodiments of the present invention, apparently, the described embodiments are a part but not all of the embodiments of the present invention. Based on the embodiments in the present invention, all the other embodiments obtained by those skilled in the art without any creative effort shall all fall within the protection scope of the present invention.

It can be known from the background art that, the existing sequence alignment methods include two stages: seed lookup and seed alignment, a series of subsequences on the read sequence to be aligned are extracted, that is, the seed, and then the candidate alignment location (CAL) table is searched, to find out the exact matching location of each seed on the reference sequence, and then the base at the matching location is read and compared with read. In order to improve the accuracy of sequence alignment, it is necessary to find out the location of the seed of the sequence to be aligned read in the reference sequence as much as possible, therefore, the length of the seed is usually shorter. However, such a seed will hit a lot of times on the reference sequence, and the performance of sequence alignment of the existing processor is limited, the sequence alignment speed is slow, and the requirement of fast or real-time acquisition of gene alignment results cannot be satisfied.

In view of this, the present invention provides a sequence alignment method and system, to solve the problems in the prior art that a seed will hit a lot of times on the reference sequence, and the performance of sequence alignment of the existing processor is limited, the sequence alignment speed is slow, and the requirement of fast or real-time acquisition of gene alignment results cannot be satisfied.

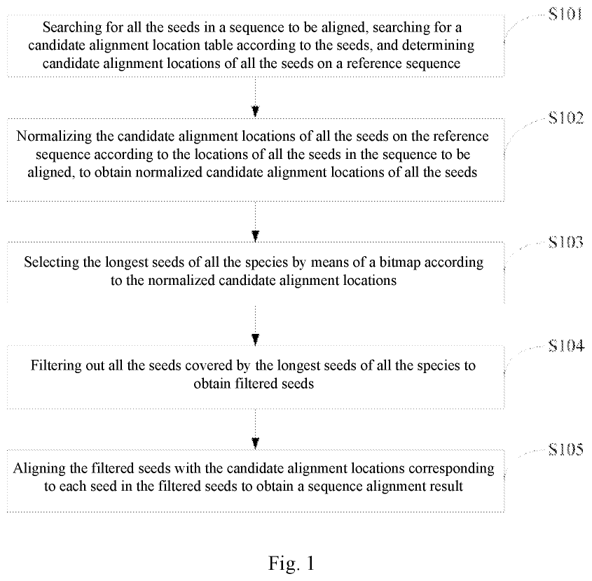

As shown in FIG. 1, the embodiment of the present invention discloses a sequence alignment method, including the following steps:

S101, searching for all the seeds in a sequence to be aligned, searching for a candidate alignment location table according to the seeds, and determining candidate alignment locations of all the seeds on a reference sequence.

It should be noted that the candidate alignment location table is pre-established before the sequence alignment process. The specific method is to shift bit by bit on the reference sequence according to the length of the seed, record its corresponding location on the reference sequence, and perform hash operation, which can reflect the location of the seed on the reference sequence.

Wherein, in the field of gene alignment, the reference sequence is a template of gene base sequence established over many years, which is also known as a standard gene library and represents the corresponding relationship between currently known genes and gene effects. Through the alignment between the sequence to be aligned and the reference sequence, the gene effect of the sequence to be aligned can be predicted. For example, the base sequence of a certain gene base represents a higher probability of suffering from a certain skin disease, and through the alignment, it can be known that the sequence to be aligned and the base sequence of the gene are completely the same or the similarity is higher than a certain level, it can be considered that the person with the sequence to be aligned has a higher probability of suffering from the skin disease.

Optionally, after determining the candidate alignment locations of all the seeds on the reference sequence, the sequence alignment method further includes:

judging whether the number of candidate alignment locations of each seed on the reference sequence exceeds a second preset threshold;

if it is judged that the number of candidate alignment locations of any seed on the reference sequence exceeds the second preset threshold, then selecting the candidate alignment locations for subsequent alignment from all the candidate alignment locations of the seed according to a preset number of intervals; and

if it is judged that the number of candidate alignment locations of each seed on the reference sequence exceeds a second preset threshold, then performing step S102.

It should be noted that, in the gene sequence alignment work, if the number of candidate alignment locations of any seed on the reference sequence is found to be too large, 1024 locations are generally taken as the second preset threshold, it means that the function of the gene base sequence represented by this seed is a certain basic function, therefore, the seed will appear for many times, and this kind of seed is of little use for disease identification, but the number of the seeds is large, so it is necessary to reduce the number of candidate alignment locations of such seeds, to improve the efficiency of the subsequent alignment work.

S102, normalizing the candidate alignment locations of all the seeds on the reference sequence according to the locations of all the seeds in the sequence to be aligned, to obtain normalized candidate alignment locations of all the seeds.

Optionally, the normalizing the candidate alignment locations of all the seeds on the reference sequence according to the locations of all the seeds in the sequence to be aligned to obtain normalized candidate alignment locations of all the seeds includes:

according to the location of all the seeds in the sequence to be aligned, normalizing the candidate alignment locations of all the seeds on the reference sequence to the candidate alignment location corresponding to the starting location of the sequence to be aligned on the reference sequence, to obtain the normalized candidate alignment location of all the seeds.

It should be noted that, through normalized operation, a complex candidate alignment location relationship can be converted into a relative relationship with the candidate alignment location of the starting location of the sequence to be aligned, to facilitate subsequent bitmap processing.

Specifically, if the location of a certain seed in the sequence to be aligned is n, and the normalized candidate alignment location is obtained after subtracting n from the candidate alignment location corresponding to the seed.

S103, selecting the longest seeds of all the species by means of a bitmap according to the normalized candidate alignment locations.

Optionally, the selecting the longest seeds of all the species by means of a bitmap according to the normalized candidate alignment locations includes:

the candidate alignment locations of all the seeds on the reference sequence are set to 1 by means of a bitmap according to the normalized candidate alignment locations, locations on the reference sequence other than the candidate alignment locations are set to 0, the longest consecutive 1 corresponding seeds at different starting locations in the bitmap are selected, to obtain the longest seeds of all the species.

It needs to be noted that, the bitmap is a representation image of a pixel array, which can intuitively express the difference in color according to the bit depth. Wherein, the present invention uses a bitmap with a bit depth of 1 for processing, and a bitmap with a bit depth of 1 has only two values of 1 and 0, which correspond to black and white respectively. The corresponding candidate alignment location of the seed on the reference sequence can be set to 1, and the location without a corresponding seed on the reference sequence is set to 0, so that the longest seed can be judged through consecutive 1.

It should be further noted that, the species of seeds refer to the seeds with different locations as the starting point. Wherein the longest seed in a certain species of seeds refers to the longest seed in all the seeds with a certain location as a starting point on the sequence to be aligned.

S104, filtering out all the seeds covered by the longest seeds of all the species to obtain filtered seeds.

It should be noted that, since the purpose of sequence alignment is to find out the sequence with the highest similarity to the sequence to be aligned on the reference sequence, and among the seeds on the sequence to be aligned where the candidate alignment location is found on the reference sequence, the longer seeds must be able to reflect higher similarity than the shorter seeds, therefore, all the seeds covered by the longest seeds at the same starting location are not required to be aligned. All the seeds covered by the longest seed are all filtered out to improve the efficiency of the subsequent alignment work.

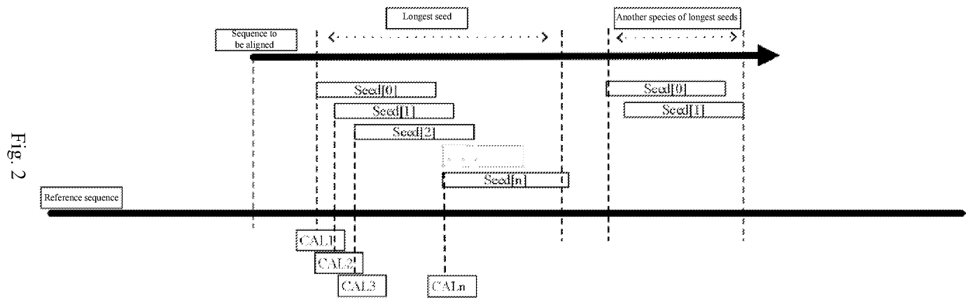

As shown in FIG. 2 which is a schematic diagram of the candidate alignment locations of the seeds in the sequence to be aligned on the reference sequence, wherein CAL is the candidate alignment sequence and Seed is the seed, it can be seen from the figure that the seeds can find the matching candidate alignment locations on the reference aligned sequence, and among them, Seed0, Seed1 and Seed2 and their corresponding candidate alignment locations are filtered out since Seed0, Seed1 and Seed2 are completely covered by the longest seeds, to improve the efficiency of subsequent alignment.

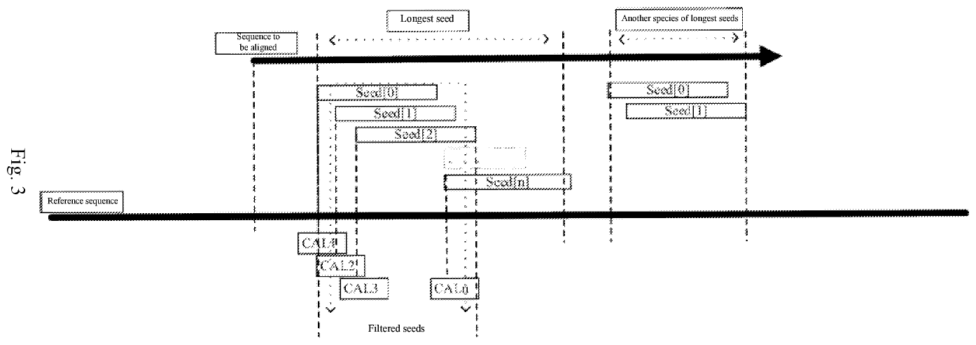

Specifically, as shown in FIG. 3, FIG. 3 is a schematic diagram of the filtered seeds.

Optionally, after filtering out all the seeds covered by the longest seeds of all the species to obtain filtered seeds, the sequence alignment method further includes:

counting the number of occurrences of each longest seed in the reference sequence;

judging whether the number of occurrences of each longest seed in the reference sequence is less than a first preset threshold;

if it is judged that the number of occurrences of any of the longest seeds in the reference sequence is less than a first preset threshold, then splitting from the longest seed a seed containing a base in the middle location of the longest seed.

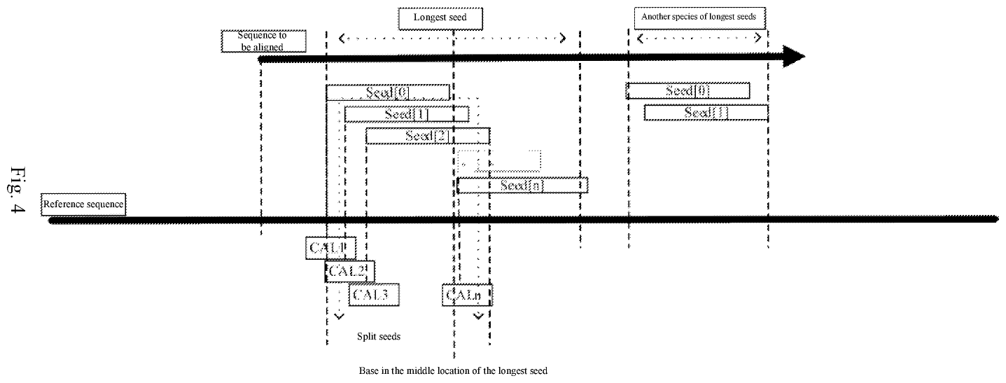

As shown in FIG. 4, FIG. 4 is a schematic diagram of seeds containing a base in the middle location of the longest seed and split from the final seeds.

If it is judged that the number of occurrences of each of the longest seeds in the reference sequence is greater than or equal to the first preset threshold, then step S105 is performed.

It should be noted that, when any of the longest seeds is found to have too few occurrences in the reference sequence, then there may be two cases, one is that there is indeed a smaller number of candidate alignment locations in the reference sequence, and the other is that the longest seed happens to be incorrectly matched to some candidate alignment locations due to gene mutation or other factors. If it is the second case, the problem of incorrect sequence alignment result will be caused when subsequent alignment is performed according to the incorrectly matched candidate alignment locations.

Therefore, aiming at the second case, a first preset threshold is set, which is generally between 20 times and 30 times, if it is judged that the number of occurrences of any longest seed in the reference sequence is less than the first preset threshold, the longest seed is considered to belong to the above second case, and the longest seed is split to obtain a plurality of seeds containing the base in the middle location of the longest seed, and sequence alignment is performed with the candidate alignment locations of these seeds on the reference sequence, to ensure the accuracy of the alignment work.

Wherein the reason why the seed split from the longest seed must contain the base in the middle location of the longest seed is that, according to the test, the base sequence in the middle location of the seed better reflects the functional effect of the gene sequence, so in order to obtain more accurate alignment results, the method that the seed split from the longest seed must contain the base in the middle location of the longest seed should be selected for splitting.

Optionally, a length threshold is set to determine whether the longest seeds of various species are too long, and if it is judged that a certain longest seed is greater than the length threshold and the number of occurrences in the reference sequence is less than a first preset threshold, it is judged that the longest seed belongs to the above second case, and a seed containing the base in the middle location of the longest seed is split from the longest seed.

It should be noted that, in general, the length of a gene sequence segment that can represent a function does not exceed a length threshold, and if the length threshold is exceeded, it is very likely that the second case occurs, i.e., gene mutation occurs. Therefore, judging whether to split the longest seed according to both the length of the longest seed and the number of occurrences on the reference sequence can further ensure the accuracy of the alignment work.

S105, aligning the filtered seeds with the candidate alignment locations corresponding to each seed in the filtered seeds to obtain a sequence alignment result.

As to the sequence alignment method disclosed in the present embodiment, the candidate alignment locations of all the seeds in the sequence to be aligned are searched for, after normalizing the candidate alignment locations of all the seeds, the longest seeds of various species are acquired through the manner of bitmap, and then all the seeds covered by the longest seeds are filtered out, to reduce the number of candidate alignment locations needing to be aligned subsequently, so as to greatly reduce the workload of subsequent alignment work, and improve the sequence alignment speed and ensure alignment accuracy.

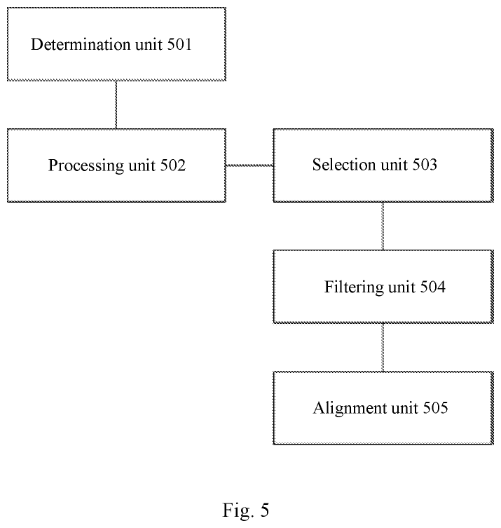

Based on the sequence alignment method disclosed in the embodiment of the present invention, FIG. 5 specifically discloses a sequence alignment system applying the sequence alignment method.

As shown in FIG. 5, another embodiment of the present invention discloses a sequence alignment system, and the system includes:

a sequence alignment system, including:

a determination unit 501, configured to search for all the seeds in a sequence to be aligned, search for a candidate alignment location table according to the seeds, and determine candidate alignment locations of all the seeds on a reference sequence;

a processing unit 502, configured to normalize the candidate alignment locations of all the seeds on the reference sequence according to the locations of all the seeds in the sequence to be aligned, to obtain normalized candidate alignment locations of all the seeds;

a selection unit 503, configured to select the longest seeds of all the species by means of a bitmap according to the normalized candidate alignment locations;

a filtering unit 504, configured to filter out all the seeds covered by the longest seeds of all the species to obtain filtered seeds; and

an alignment unit 505, configured to align the filtered seeds with the candidate alignment locations corresponding to each seed in the filtered seeds to obtain a sequence alignment result.

Optionally, the selection unit 503 is configured to set the candidate alignment locations of all the seeds on the reference sequence to 1 by means of a bitmap according to the normalized candidate alignment locations, set locations on the reference sequence other than the candidate alignment locations to 0, select the longest consecutive 1 corresponding seeds at different starting locations in the bitmap, to obtain the longest seeds of all the species.

Optionally, the sequence alignment system further includes:

a statistical unit, configured to count the number of occurrences of each longest seed in the reference sequence; and

a first judgment unit, configured to judge whether the number of occurrences of each longest seed in the reference sequence is less than a first preset threshold;

if it is judged that the number of occurrences of any of the longest seeds in the reference sequence is less than a first preset threshold, then splitting from the longest seed a seed containing a base in the middle location of the longest seed; and

if it is judged that the number of occurrences of each of the longest seeds in the reference sequence is greater than or equal to the first preset threshold, then performing the step of aligning the filtered seeds with the candidate alignment locations corresponding to each seed in the filtered seeds to obtain the sequence alignment results.

Optionally, the processing unit 502 is further configured to, according to the locations of all the seeds in the sequence to be aligned, normalize the candidate alignment locations of all the seeds on the reference sequence to a candidate alignment location on the reference sequence corresponding to the starting location of the sequence to be aligned to obtain the normalized candidate alignment locations of all the seeds.

Optionally, the sequence alignment system further includes:

a second judgment unit, configured to judge whether the number of candidate alignment locations of each seed on the reference sequence exceeds a second preset threshold;

if it is judged that the number of candidate alignment locations of any seed on the reference sequence exceeds the second preset threshold, then selecting the candidate alignment locations for subsequent alignment from all the candidate alignment locations of the seed according to a preset number of intervals;

if it is judged that the number of candidate alignment locations of each seed on the reference sequence exceeds a second preset threshold, then performing the step of normalizing the candidate alignment locations of all the seeds on the reference sequence according to the locations of all the seeds in the sequence to be aligned, to obtain the normalized candidate alignment locations of all the seeds.

As to the specific working process of the determination unit 501, the processing unit 502, the selection unit 503, the filtering unit 504 and the alignment unit 505 in the sequence alignment system disclosed in the above embodiment of the present invention, please refer to the corresponding contents of the sequence alignment method disclosed in the above embodiment of the present invention, which will not be repeated redundantly herein.

As to the sequence alignment system disclosed in the present embodiment, the candidate alignment locations of all the seeds in the sequence to be aligned are searched for, after normalizing the candidate alignment locations of all the seeds, the longest seeds of various species are acquired through the manner of bitmap, and then all the seeds covered by the longest seeds are filtered out, to reduce the number of candidate alignment locations needing to be aligned subsequently, so as to greatly reduce the workload of subsequent alignment work, and improve the sequence alignment speed and ensure alignment accuracy.

It should also be noted that, the terms “include”, “comprise”, or any other variation thereof are intended to cover non-exclusive inclusion, such that a process, method, article, or device including a set of elements includes not only those elements, but also other elements not expressly listed or also includes elements inherent to such process, method, article, or device. Without further limitation, the inclusion of an element as defined by the statement “including a . . . ” does not preclude the existence of additional identical elements in the process, method, article, or device including the element.

Those skilled in the art should understand that, the embodiment of the present application can be provided as a method, a system or a computer program product. Therefore, the present application may adopt the form of a complete hardware embodiment, a complete software embodiment, or an embodiment combining software and hardware. Moreover, the present application may adopt the form of a computer program product implemented on one or more computer usable storage media (including but not limited to disk storage, CD-ROM, optical storage, etc.) containing computer usable program codes.

The above are only embodiments of the present application, and are not used to limit the present application. For those skilled in the art, various modifications and variations may be made to the present application. Any modification, equivalent substitution, improvement and the like made within the spirit and principle of the present application shall be included in the scope of the claims of the present application.

1. A sequence alignment method, comprising:
searching for all seeds in a sequence to be aligned, searching for a candidate alignment position table according to the seeds, and determining candidate alignment positions of all the seeds on a reference sequence;
normalizing the candidate alignment positions of all the seeds on the reference sequence according to the positions of all the seeds in the sequence to be aligned, to obtain normalized candidate alignment positions of all the seeds;
selecting the longest seeds of all the species by means of a bitmap according to the normalized candidate alignment positions;
filtering out all the seeds covered by the longest seeds of all the species to obtain filtered seeds; and
aligning the filtered seeds with the candidate alignment positions corresponding to each seed in the filtered seeds to obtain a sequence alignment result.

2. The sequence alignment method according to claim 1, wherein, the selecting the longest seeds of all the species by means of a bitmap according to the normalized candidate alignment positions comprises:
setting the candidate alignment positions of all the seeds on the reference sequence to 1 by means of a bitmap according to the normalized candidate alignment positions, setting positions on the reference sequence other than the candidate alignment positions to 0, selecting the longest consecutive 1 corresponding seeds at different starting positions in the bitmap, to obtain the longest seeds of all the species.

3. The sequence alignment method according to claim 1, wherein, after filtering out all the seeds covered by the longest seeds of all the species to obtain filtered seeds, the sequence alignment method further comprises:
counting the number of occurrences of each longest seed in the reference sequence;
judging whether the number of occurrences of each longest seed in the reference sequence is less than a first preset threshold;
if it is judged that the number of occurrences of any of the longest seeds in the reference sequence is less than a first preset threshold, then splitting from the longest seed a seed containing a base at the middle position of the longest seed; and
if it is judged that the number of occurrences of each of the longest seeds in the reference sequence is greater than or equal to the first preset threshold, then performing the step of aligning the filtered seeds with the candidate alignment positions corresponding to each seed in the filtered seeds to obtain the sequence alignment results.

4. The sequence alignment method according to claim 1, wherein, the normalizing the candidate alignment positions of all the seeds on the reference sequence according to the positions of all the seeds in the sequence to be aligned to obtain normalized candidate alignment positions of all the seeds comprises:
according to the positions of all the seeds in the sequence to be aligned, normalizing the candidate alignment positions of all the seeds on the reference sequence to a candidate alignment position on the reference sequence corresponding to the starting position of the sequence to be aligned to obtain the normalized candidate alignment positions of all the seeds.

5. The sequence alignment method according to claim 1, wherein, after determining the candidate alignment positions of all the seeds on the reference sequence, the sequence alignment method further comprises:
judging whether the number of candidate alignment positions of each seed on the reference sequence exceeds a second preset threshold;
if it is judged that the number of candidate alignment positions of any seed on the reference sequence exceeds the second preset threshold, then selecting the candidate alignment positions for subsequent alignment from all the candidate alignment positions of the seed according to a preset number of intervals; and
if it is judged that the number of candidate alignment positions of each seed on the reference sequence exceeds a second preset threshold, then performing the step of normalizing the candidate alignment positions of all the seeds on the reference sequence according to the positions of all the seeds in the sequence to be aligned to obtain the normalized candidate alignment positions of all the seeds.

6. (canceled)

7. (canceled)

8. (canceled)

9. (canceled)

10. (canceled)
